(19) United States
(12) Patent Application Publication
(10) Pub. No.: US 2007/0185209 A1
(43) Pub. Date: Aug. 9, 2007

(54) Treatment methods using triaryl methane compounds

(75) Inventors: Neil A. Castle, Cary, NC (US); Gregory C. Rigdon, Durham, NC (US); Douglas S. Krafte, Carrboro, NC (US)

Correspondence Address: MORGAN, LEWIS & BOCKIUS LLP (SF), 2 PALO ALTO SQUARE, 3000 El Camino Real, Suite 700, PALO ALTO, CA, 94306, US

(73) Assignee: Icagen, Inc., Durham, NC (US)

(21) Appl. No.: 11/642,416

(22) Filed: Dec. 20, 2006

Related U.S. Application Data
(60) Provisional application No. 60/752,935, filed on Dec. 20, 2005.

(51) Int. Cl. A61K 31/165

(52) U.S. Cl. 514/617

(57) ABSTRACT

The use of novel inhibitors of potassium flux is disclosed for the treatment of inflammatory processes, such as multiple sclerosis, insulin-dependent (type I) diabetes mellitus, rheumatoid arthritis, peripheral neuritis and pulmonary hypertension. The compounds are also of use in treating and preventing stroke. These inhibitors have a high specificity for the IK1 channel and greater stability relative to non-fluorine substituted homologues.

### CROSS-REFERENCE TO RELATED APPLICATIONS

This application claims the benefit of prior U.S. Provisional Application Serial No. 60/752,935, filed on Dec. 20, 2005, the disclosure of which is incorporated herein in its entirety for all purposes.

### BACKGROUND OF THE INVENTION

In one aspect the present invention is directed to a method for treating or preventing an inflammatory process which includes, among others, multiple sclerosis and pulmonary hypertension.

Multiple sclerosis (MS) is a chronic inflammatory disease of the central nervous system. Individuals affected by MS present neurological deficits including loss of vision, motor deterioration, sensory impairment, incontinence, and other issues related to defects in the central nervous system; however, MS does not impair cognitive function. MS disease progression has a highly variable course with persons experiencing acute symptoms followed by periods of remission and then later progression to a chronic and degenerative condition. The precise cause of MS is unknown, however some speculate it may be a combination of autoimmunity, genetics, environmental factors and/or viral infections. Evidence suggests that the earlier phase of MS may be caused by autoimmune reactions, while the later chronic phases may be attributed to degeneration of the myelin sheath and the underlying axon. Steinman, Nature Immunology, 2: 762-764 (2001). Consistent with this theory, recent studies in murine models suggest that activated CD4+T-cells contribute to MS pathogenesis by attacking the central nervous system. Thus, the suppression of autoreactive T-cells represents a potential therapeutic avenue. Beeton, C. et al., PNAS, 98: 13942-13947 (2001); Wulff, et al, J. Clin. Invest., 111: 1703-1713 (2003).

T-cells become activated by the influx of Ca²⁺ions via Ca²⁺release-activated Ca²⁺(CRAC) channels. To counterbalance the influx of positive ions, two K⁺ channels, Kv1.3 and IK1 operate to push K⁺ out of the cell. These K⁺ channels operate by two distinct mechanisms. Kv1.3 is a voltage-gated channel that opens in response to membrane depolarization and operates to maintain a resting membrane potential. Beeton, et al., PNAS, 98: 13942-13947 (2001). IK1 responds to an increase in cytosolic Ca²⁺and operates to hyperpolarize the membrane potential. Taken together, both channels play important roles in T-cell activation, adhesion, and migration. In T-cells both the mRNA and protein expression of IK1 and Kv1.3 channels is upregulated in response to antigenic and mitogenic stimuli.

These potassium channels are good targets for drug candidates because of their restricted expression. They are predominantly expressed in blood and epithelial cells. The specific expression pattern of these channels suggests that specific K⁺-channel inhibitors may have fewer side-effects.

Experimentally, several known inhibitors selectively block Kv1.3 and IK1 channels over other K⁺-channels. A peptide toxin from Stichodactyla helianthus (ShK) inhibits both Kv1.3 and IK1 channels. In vitro studies show that Shk-Dap²², margatoxin, and correolide specifically inhibit the Kv1.3 channel while clotrimazole and TRAM-4 (a synthetic analog of clotrimazole) specifically inhibit IK1. Ghanshani, et al., J. Biol. Chem. 275: 37137-37149 (2000). Using in vitro studies, Beeton et al showed that selective Kv1.3 blockers inhibited proliferation in chronically activated T-cells, while selective IK1 blockers inhibited proliferation in acutely activated T-cells. Beeton, et al., PNAS, 98: 13942-13947 (2001).

Experimental autoimmune encephalomyelitis (EAE) mice mimic many of the pathological features of MS and are widely studied as the standard animal model. Laboratory animals depleted of T-cells exhibit a loss of ability to develop EAE, suggesting T-cells are necessary for development of MS in humans. In an adoptive-transfer EAE animal model, Beeton et al identified that the more general potassium channel blocker, ShK, provided the most potent treatment to prevent the lethal EAE adoptive transfer and ameliorate disease progression. The Kv1.3 specific blocker Shk-Dap²² , offered the second best protection, while the IK1 specific blocker TRAM-34, offered the least effective treatment. Of note, the combination of Shk-Dap²² and TRAM-34 offered greater protection than Shk-Dap²² alone. This result could be potentially explained by the high ratio of Kv1.3 channels compared to IK1 channels in chronically activated T-cells. Indeed, myelin-reactive T-cells taken from MS patients also contain a high Kv1.3 compared to IK1 ratio, suggesting that these cells undergo multiple rounds of antigen stimulation during disease progression.

In contrast to the adoptive transfer murine model described above, it has been found that TRAM-34 reduced the development of EAE in mice immunized with a peptide fragment of the myelin oligodendrocyte glycoprotein. Madsen, et al, Eur. J. Immunol. 35: 10 (2005); Reich, et al., Eur. J. Immunol., 35: 1 (2005). Interestingly, this study showed that TRAM-34 had no effect on T-cell clonal expansion, but it did strongly reduce cytokine expression levels. These in vivo studies suggest that Kv1.3 and IK1 channels do not have redundant characteristics. Thus, the development and testing of novel K⁺-channel blockers may provide additional information for understanding molecular mechanisms and treating the disease.

Certain presently known IK1 inhibitors have several problems. Clotrimazole and other related antimycotic agents including miconazole, econoazole, butoconazole, oxiconazole and sulconazole have been shown to inhibit IK1 and prevent loss of K⁺, they are not ideal clinical drugs due to potential and observed hepatotoxicity. They also have low in vivo half lives, low bioavailabilities and a relatively low potency in their interaction with IK1. Some inhibitors have non-specific interactions with non-IK1 calcium activated potassium channels. Thus, there remains a need for IK1 channel inhibitors. The present invention describes a group of select IK1 channel inhibitors that fulfills these and other needs.

The invention is particularly useful in treating or preventing pulmonary hypertension. Thus the present invention provides a method of treating or preventing pulmonary hypertension. The method includes administering to a subject suffering from pulmonary hypertension a therapeutically effective amount of a compound having the structure according to Formula (I). This method is particularly useful in those subjects who additionally suffer from sickle cell disease.

Pulmonary hypertension (or PH) as used herein refers to an abnormal elevation of the pressure in the blood vessels in the lungs, the pulmonary arteries. Over time, the increased pressure damages both the large and small pulmonary arteries. The walls of the smallest blood vessels thicken and are no longer able to transfer oxygen and carbon dioxide normally between the blood and the lungs. Thus, the levels of oxygen in the blood may fall. The low oxygen level can cause narrowing (constriction) of the pulmonary arteries. These changes contribute further to the increased pressure in the pulmonary circulation.

With pulmonary hypertension, the right side of the heart must work harder to push the blood through the pulmonary arteries into the lungs. Over time, the right ventricle becomes thickened and enlarged, leading to a condition called cor pulmonale.

In some people, the bone marrow produces more red blood cells to compensate for less oxygen in the blood, leading to a condition called polycythemia. The extra red blood cells cause the blood to become thicker and stickier, further increasing the load on the heart. These changes also put a person with cor pulmonale at increased risk of pulmonary embolism, because the thickened blood may clump and form clots, mainly in the veins of the legs. These clots can dislodge and travel to the lungs.

There are two types of pulmonary hypertension: primary and secondary. Both types of pulmonary hypertension are encompassed by this term as used herein. Primary pulmonary hypertension is much less common than secondary pulmonary hypertension. In primary pulmonary hypertension, the cause is not known, but likely begins with spasm (contraction) of the muscle layer in the pulmonary arteries. Women are affected by primary pulmonary hypertension twice as often as men, and half of the people are 35 or older at the time of diagnosis. Secondary pulmonary hypertension means that the condition occurred because of another disorder that affects lung structure or function.

Secondary pulmonary hypertension can be caused by any disease that impedes the flow of blood through the lungs or that causes sustained periods of low oxygen in the blood. One of the most common causes is chronic obstructive pulmonary disease. When the lungs are impaired by disease, it takes more effort to pump blood through them. Over time, chronic obstructive pulmonary disease destroys the small air sacs (alveoli) together with their small vessels (capillaries) in the lungs. The single most important cause of pulmonary hypertension in chronic obstructive pulmonary disease is the narrowing of the pulmonary artery that occurs as a result of low blood oxygen levels.

Another disease that can cause pulmonary hypertension is pulmonary fibrosis, which causes extensive scar tissue to form in the lungs. The scar tissue destroys the pulmonary circulation and makes blood flow more difficult. Other lung diseases that may cause pulmonary hypertension include cystic fibrosis and certain occupational lung diseases, such as asbestosis and silicosis.

Less often, pulmonary hypertension is caused by extensive loss of lung tissue from surgery or trauma, or by heart failure, scleroderma, obesity with reduced ability to breathe (Pickwickian syndrome), neurologic diseases involving the respiratory muscles, chronic liver disease, HIV infection, and diet drugs. A sudden cause of pulmonary hypertension is pulmonary embolism, a condition in which blood clots become lodged in the arteries of the lung, causing serious problems.

Some people with pulmonary hypertension have connective tissue disorders, especially scleroderma. When people have both conditions, Raynaud's phenomenon often develops before symptoms of pulmonary hypertension appear.

Reducing T-cell activation via blockade of the Kv1.3 and/or the IK1 channel is an approach towards the treatment and/or prevention of inflammatory processes. Compounds capable of inhibiting the Kv1.3 and/or the IK1 channel as a means of reducing inflammation are therefore desirable. Although of demonstrable efficacy, the imidazole-based Kv1.3 and/or the IK1 channel inhibitors that have been explored to date are hampered by several shortcomings including a well-documented potential for hepatotoxicity. This toxicity is exacerbated by the inhibitors' low potencies, non-specific interactions with calcium activated potassium channels other than the Kv1.3 and/or the IK1 channel and low bioavailabilities, each of which motivate for the administration of higher and more frequent dosages of the inhibitors.

### SUMMARY OF THE INVENTION

Triphenylacetamide-based K⁺-channel blockers are promising candidates for the treatment of sickle cell disease (SCD) as discussed in U.S. Pat. No. 6,288,122 which is herein incorporated by reference. In addition, studies indicate that triphenylacetamide-based inhibitors are potential candidate drugs for the treatment of inflammatory conditions, such as MS or PH. In vitro studies show that a triphenylacetamide-based inhibitor, compound 3 in Table 1, which has a long half-life, inhibits K⁺ channels with a high selectivity for the IK1 channel.

Thus, in a first aspect, the present invention provides a method for treating or preventing an inflammatory process, said method comprising administering to a subject suffering from said inflammatory process a therapeutically effective amount of a compound having the structure according to Formula I: 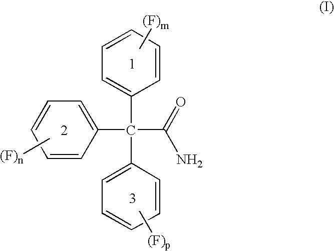
wherein m, n and p are independently selected from 0 and 1 and at least one of m, n and p is 1.

In an exemplary embodiment, when m, n and p are all 1, the fluoro substituents at ring 1 and at ring 2 are located at a position independently selected from ortho to the acetamide substituent, meta to the acetamide substituent and para to the acetamide substituent, and the substituent at ring 3 is at a position selected from ortho to the acetamide substituent and para to the acetamide substituent. In another exemplary embodiment, when p is 0, and m is 1 and n is 1, the fluoro substituent at ring 1 is para to the acetamide substituent, and the substituent at ring 2 is located at a position selected from ortho to the acetamide substituent and para to the acetamide substituent.

Controlling inflammatory processes (e.g., multiple sclerosis) via altering cellular ionic fluxes of cells affected by a disease is a powerful therapeutic approach. Moreover, basic understanding of the role of cellular ionic fluxes in both disease processes and normal physiology promises to provide new therapeutic modalities, regimens and agents. Compounds that alter cellular ion fluxes, particularly those that inhibit potassium flux, are highly desirable as both drugs and as probes for elucidating the basic mechanisms underlying these ion fluxes. Similarly, methods utilizing these compounds in basic research and in therapeutic applications are valuable tools in the arsenal of both the researcher and clinician. Therefore such compounds and methods are also an object of the present invention.

Thus, in another aspect, the present invention provides a method of inhibiting potassium flux of a cell. The method includes contacting a cell with an amount of a compound according to Formula (I) effective to inhibit the potassium flux.

An important therapeutic pathway for treatment of an inflammatory process, such as multiple sclerosis is preventing or retarding autoreactive T-cell growth. This growth retardation can be accomplished by manipulating the cellular ion fluxes of the T-cells. Thus, in another aspect, the invention provides a method for preventing or retarding autoreactive T-cell growth. The method includes contacting a T-cell with an amount of a compound according to Formula (I) effective for preventing or retarding autoreactive T-cell growth.

Thus, in another aspect, the present invention provides for a method of treating or preventing multiple sclerosis. The method includes administering to a subject suffering from multiple sclerosis a therapeutically effective amount of a compound having a structure according to Formula (I). In another exemplary embodiment, the method involves treating multiple sclerosis by administering a compound of the invention to a mammal not otherwise in need of treatment treatment with the compounds of the invention.

In another aspect, the present invention provides a method of treating or preventing pulmonary hypertension. The method includes administering to a subject suffering from pulmonary hypertension a therapeutically effective amount of a compound having the structure according to Formula (I). In another exemplary embodiment, the method involves treating pulmonary hypertension by administering a compound of the invention to a mammal not otherwise in need of treatment with the compounds of the invention.

In still a further aspect, the invention provides a method of treating or preventing stroke. The method includes administering to a subject suffering from stroke, or at risk of having a stroke, a therapeutically effective amount of a compound having the structure according to Formula (I). There is an excellent track record of treating nervous and cardiovascular disorders with ion channel modulators—either openers or blockers. Ion channel blockers as a general class, represent the major therapeutic agents for treatment of stroke, epilepsy and arrhythmias. In another exemplary embodiment, the method involves treating or preventing stroke by administering a compound of the invention to a mammal not otherwise in need of treatment with the compounds of the invention.

These and other objects and advantages of the present invention will be apparent from the detailed description and examples that follow.

### DETAILED DESCRIPTION OF THE INVENTION

Abbreviations and Definitions

“Biological medium,” as used herein refers to both in vitro and in vivo biological milieus. Exemplary in vitro “biological media” include, but are not limited to, cell culture, tissue culture, homogenates, plasma and blood. In vivo applications are generally performed in mammals, preferably humans.

“Fluoroalkyl” refers to a subclass of “substituted alkyl” encompassing alkyl or substituted alkyl groups that are either partially fluorinated or per-fluorinated. The fluorine substitution can be the only substitution of the alkyl moiety or it can be in substantially any combination with any other substituent or group of substituents.

Compounds

In a first aspect, the present invention utilizes a compound having a structure according to Formula (I): 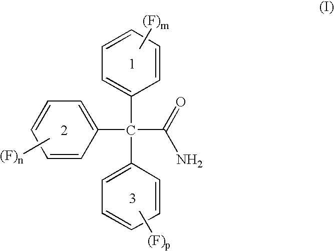
wherein m, n and p are independently selected from 0 and 1 and at least one of m, n and p is 1.

In an exemplary embodiment, when m, n and p are all 1, the fluoro substituents at ring 1 and at ring 2 are located at a position independently selected from ortho to the acetamide substituent, meta to the acetamide substituent and para to the acetamide substituent, and the substituent at ring 3 is at a position selected from ortho to the acetamide substituent and para to the acetamide substituent. In another exemplary embodiment, when p is 0, and m is 1 and n is 1, the fluoro substituent at ring 1 is para to the acetamide substituent, and the substituent at ring 2 is located at a position selected from ortho to the acetamide substituent and para to the acetamide substituent.

In an exemplary embodiment, the compounds utilized in the present invention have a structure according to Formula (II): 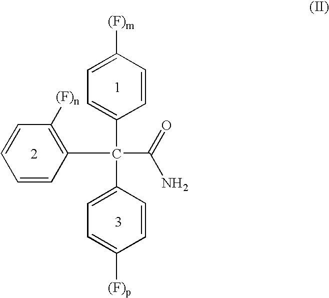
wherein m, n and p are independently selected from 0 and 1, and at least one of m, n and p is 1.

Compounds according to this structure are displayed in Table 1.

In another exemplary embodiment, the compounds of the invention have a structure according to Formula III: 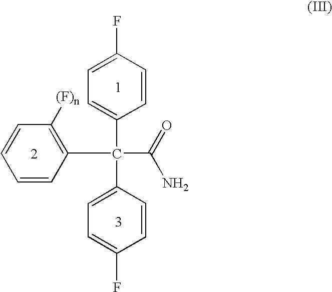
wherein n is either 0 or 1.

Compounds that are structurally closely related to compounds of the invention are also displayed in Table 1. The compounds which are structurally related to the compounds of the invention serve as a “baseline” for assessing the advantages and unexpected properties and benefits of the fluorinated compounds of the invention.

TABLE 1

| | |
|---|---|
| | (1) |
| 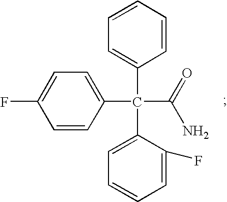 | |
| 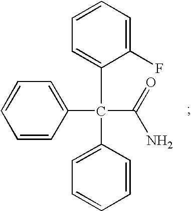 | (2) |
| 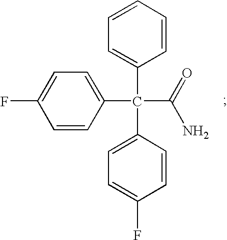 | (3) |
| 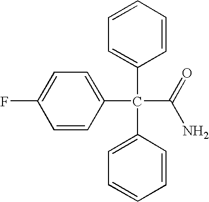 | (4) |
| 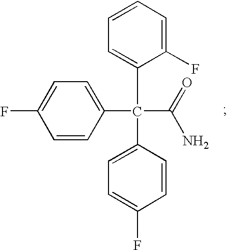 | (5) |
| 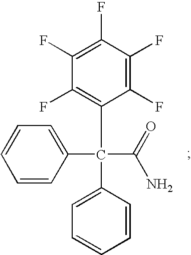 | (6) |
| 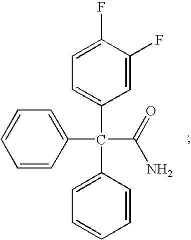 | (7) |
| 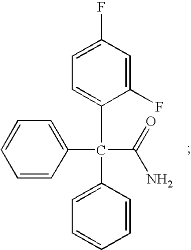 | (8) |
| 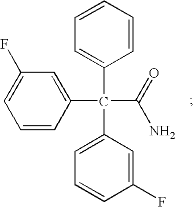 | (9) |
| 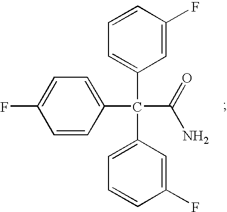 | (10) |
| 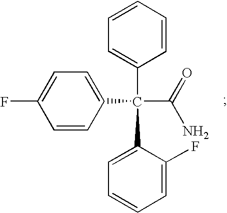 | (11) |
| 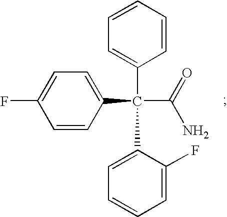 | (12) |
| 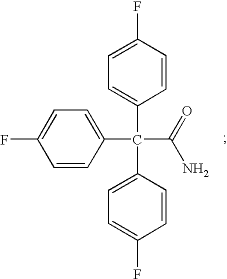 | (13) |
| 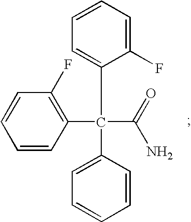 | (14) |
| 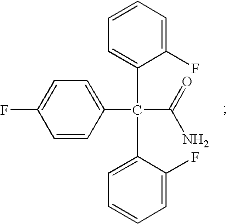 | (15) |
| 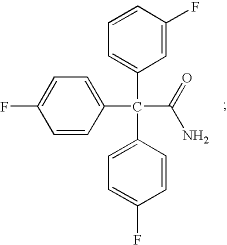 | (16) |
| 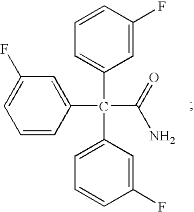 | (17) |
| 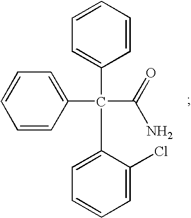 | (18) |
| 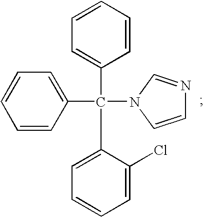 | (19) |
| 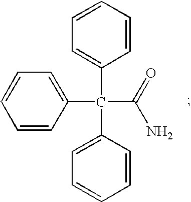 | (20) |
| 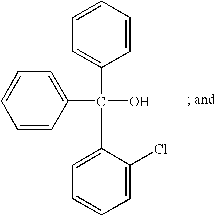 | (21) |
| 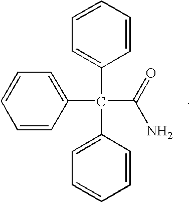 | (22) |

Compound Synthesis

The compounds of the invention can be prepared by techniques that are standard in the art of organic synthesis. Appropriate starting materials and reagents can be obtained commercially or they can be prepared by standard organic chemistry techniques. Exemplary processes are illustrated by the specific examples. An exemplary synthetic route is provided in Scheme 1.

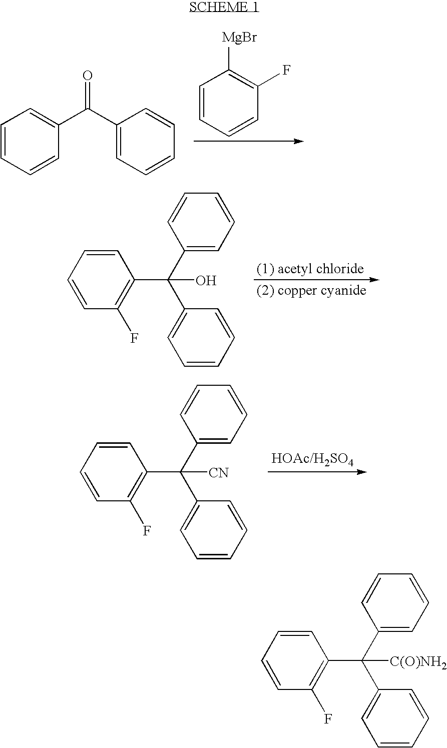

In Scheme 1, the synthesis of a fluorine-substituted triphenylacetamide proceeds from the corresponding fluorine-substituted triphenylmethanol that is prepared from a fluorine-substituted benzophenone and a reagent that adds a phenyl or fluorine-substituted phenyl moiety to the benzophenone ketone. The fluorine-substituted triphenylmethanol is subsequently converted to the corresponding fluorine-substituted triphenylacetonitrile by exposing the alcohol to acetyl chloride followed by copper cyanide. The acetamide can be formed by reacting the intermediate nitrile with a mixture of sulfuric and glacial acetic acids. Other synthetic routes leading to fluorine-substituted triphenylmethane species, particularly acetamides, are within the abilities of those skilled in the art.

Compound Stability

For compounds to act as pharmaceutically useful IK1 channel inhibitors, candidate compounds must demonstrate both acceptable bioavailability and stability in vivo. Subjects undergoing treatment must be regularly dosed with the compound of the invention. Compounds having increased in vivo residence times and increased bioavailability allow for a simplified dosage regimen (i.e. fewer doses/day and/or less medication). Moreover, reducing the amount of compound administered carries with it the promise of reducing side effects resulting from the medication and/or its metabolites. Thus, it is highly desirable to provide IK1 channel inhibitors which demonstrate good bioavailability and enhanced in vivo stability.

Compound Activity

To develop pharmaceutically useful IK1 channel inhibitors, candidate compounds must demonstrate acceptable activity towards the target channel. Compounds are judged to be sufficiently potent if they have an IC50 towards the IK1 channel of no more than 100-500 nM.

The activity of the compounds of the invention towards ion channels can be assayed utilizing methods known in the art. For example, see, Brugnara et al., J. Biol. Chem., 268(12): 8760-8768 (1993). Utilizing the methods described in this reference, both the percent inhibition of the Gardos channel and the IC50 of the compounds of the invention can be assayed.

Other methods for assaying the activity of ion channels and the activity of 30 agents that affect the ion channels are known in the art. The selection of an appropriate assay methods is well within the capabilities of those of skill in the art. See, for example, Hille, B., Ionic Channels Of Excitable Membranes, Sinaner Associates, Inc., Sunderland, Mass. (1992).

Compound Selectivity

For compounds to act as pharmaceutically useful IK1 channel inhibitors, candidate compounds must demonstrate acceptable selectivity towards the target channel. Compounds having a selectivity towards the Gardos channel of at least 30 fold are judged to be sufficiently selective.

The selectivity of a particular compound for the IK1 channel relative to another potassium ion channel is conveniently determined as a ratio of two compound binding-related quantities (e.g., IC50). In an exemplary embodiment, the selectivity is determined using the activities determined as discussed above, however, other methods for assaying the activity of ion channels and the activity of agents that affect the ion channels are known in the art. The selection of appropriate assay methods is well within the capabilities of those of skill in the art. See, for example, Hille, B., Ionic Channels Of Excitable Membranes, Sinaner Associates, Inc., Sunderland, Mass. (1992).

In one embodiment, the compounds of the invention are potent, selective and stable inhibitors of potassium flux, such as that mediated by the IK1 channel.

While not wishing to be bound by any particular theory of operation, it is presently believed that certain structural features of the compounds of the invention (i.e. replacement of hydrogen with fluorine) are presently implicated in the stability, selectivity and potency of these compounds. Thus, in an exemplary embodiment, the inhibitors of the invention include an aryl moiety, wherein at least one hydrogen atom of the aryl moiety is replaced by a radical comprising a fluorine atom. In this embodiment, the invention encompasses fluorinated derivatives of compounds that inhibit potassium ion flux, particularly those having IK1 channel inhibitory activity (e.g., antimycotic agents, e.g., miconazole, econazole, butoconazole, oxiconazole and sulconazole). Other agents that have potassium ion channel inhibitory activity, and particularly IK1 channel inhibitory activity, and possess at least one aryl moiety bearing at least one fluorine atom are within the scope of the present invention.

In an exemplary embodiment, the aryl moiety is a phenyl group. In another exemplary embodiment, the aryl moiety is a constituent of a triphenylmethyl group.

The compound(s) of the invention can be administered per se or in the form of a pharmaceutical composition wherein the active compound(s) is in admixture with one or more pharmaceutically acceptable carriers, excipients or diluents. Thus, in addition to compounds that affect cellular ion fluxes (e.g., IK1 channel inhibiting activity), the present invention also provides pharmaceutical formulations that contain the compounds of the invention.

Pharmaceutical Formulations

In a second aspect, the invention provides a pharmaceutical formulation comprising a compound of the invention according to Formula (I) admixed with a pharmaceutically acceptable excipient. In an exemplary embodiment, the compounds are those according to Formula (II) and more preferably according to Formula (III).

The compounds described herein, or pharmaceutically acceptable addition salts or hydrates thereof, can be formulated so as to be delivered to a patient using a wide variety of routes or modes of administration. Suitable routes of administration include, but are not limited to, inhalation, transdermal, oral, ocular, rectal, transmucosal, intestinal and parenteral administration, including intramuscular, subcutaneous and intravenous injections.

The compounds described herein, or pharmaceutically acceptable salts and/or hydrates thereof, may be administered singly, in combination with other compounds of the invention, and/or in cocktails combined with other therapeutic agents. The choice of therapeutic agents that can be co-administered with the compounds of the invention will depend, in part, on the condition being treated.

For example, when administered to patients suffering from an inflammatory process such as multiple sclerosis, the compounds of the invention can be administered in cocktails containing agents used to treat the pain, infection and other symptoms and side effects commonly associated with an inflammatory process. Such agents include, e.g. analgesics, antibiotics, etc. The compounds can also be administered in cocktails containing other agents that are commonly used in treating inflammatory process, including butyrate and butyrate derivatives (Perrine et al., N. Engl. J. Med. 328(2): 81-86 (1993)); hydroxyurea (Charache et al., N. Engl. J. Med. 323(20): 1317-1322 (1995)); erythropoietin (Goldberg et al, N. Engl. J. Med. 323(6): 366-372 (1990)); and dietary salts such as magnesium (De Franceschi et al., Blood 88(648a): 2580(1996)).

Pharmaceutical compositions for use in accordance with the present invention can be formulated in a conventional manner using one or more physiologically acceptable carriers comprising excipients and auxiliaries, which facilitate processing of the active compounds into preparations which can be used pharmaceutically. Proper formulation is dependent upon the route of administration chosen.

For injection, the agents of the invention can be formulated in aqueous solutions, preferably in physiologically compatible buffers such as Hanks's solution, Ringer's solution, or physiological saline buffer. In an exemplary embodiment, the formulation includes water and an alcohol and/or glycol. Other useful components of this formulation include, for example, surfactant, emulsifiers and materials such as ethoxylated oils. An exemplary formulation includes a compound of the invention, poly(ethyleneglycol) 400, ethanol and water in a 1:1:1 ratio. Another exemplary formulation includes a compound of the invention, water, poly(ethyleneglycol) 400 and Cremophor-EL.

For transmucosal administration (e.g., buccal, rectal, nasal, ocular, etc.), penetrants appropriate to the barrier to be permeated are used in the formulation. Such penetrants are generally known in the art.

For oral administration, the compounds can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be combined with a solid excipient, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents may be added, such as the cross-linked polyvinyl pyrrolidone, agar, or alginic acid or a salt thereof such as sodium alginate.

Pharmaceutical formulations for parenteral administration include aqueous solutions of the active compounds in water-soluble form, such as those described above for intravenous administration. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation or transcutaneous delivery (e.g., subcutaneously or intramuscularly), intramuscular injection or a transdermal patch. Thus, for example, the compounds may be formulated with suitable polymeric or hydrophobic materials (e.g., as an emulsion in an acceptable oil) or ion exchange resins, or as sparingly soluble derivatives, for example, as a sparingly soluble salt.

The pharmaceutical compositions also may include suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Effective Dosages

Pharmaceutical compositions suitable for use with the present invention include compositions wherein the active ingredient is contained in a therapeutically effective amount, i.e., in an amount effective to achieve its intended purpose. The actual amount effective for a particular application will depend, inter alia, on the condition being treated. For example, when administered in methods to reduce the occurrence of multiple sclerosis and/or impair the formation of autoreactive T-cells, such compositions will contain an amount of active ingredient effective to achieve this result. Determination of an effective amount is well within the capabilities of those skilled in the art, especially in light of the detailed disclosure herein.

For any compound described herein, the therapeutically effective amount can be initially determined from cell culture assays. Target plasma concentrations will be those concentrations of active compound(s) that are capable of inducing inhibition of the IK1 channel. In exemplary embodiments, the IK1 channel activity is at least 25% inhibited. Target plasma concentrations of active compound(s) that are capable of inducing at least about 50%, 75%, or even 90% or higher inhibition of the IK1 channel potassium flux are presently preferred. The percentage of inhibition of the IK 1 channel in the patient can be monitored to assess the appropriateness of the plasma drug concentration achieved, and the dosage can be adjusted upwards or downwards to achieve the desired percentage of inhibition.

As is well known in the art, therapeutically effective amounts for use in humans can also be determined from animal models. For example, a dose for humans can be formulated to achieve a circulating concentration that has been found to be effective in animals. A particularly useful animal model for multiple sclerosis is the EME mouse model (Beeton, et al., PNAS, 98: 13942-13947 (2001); Reich, et al, Eur. J. Immunol., 35: 1 (2005); Lars Madsen, et al., Eur. J. Immunol. 35: 10 (2005). The dosage in humans can be adjusted by monitoring IK1 channel inhibition and adjusting the dosage upwards or downwards, as described above.

Adjusting the dose to achieve maximal efficacy in humans based on the methods described above and other methods as are well-known in the art is well within the capabilities of the ordinarily skilled artisan. In the case of local administration, the systemic circulating concentration of administered compound will not be of particular importance. In such instances, the compound is administered so as to achieve a concentration at the local area effective to achieve the intended result.

Patient doses for oral administration of the compounds described herein, which is the preferred mode of administration for prophylaxis and for treatment of inflammatory process episodes, typically range from about 1 mg/day to about 10,000 mg/day, more typically from about 10 mg/day to about 1,000 mg/day, and most typically from about 50 mg/day to about 500 mg/day. Stated in terms of patient body weight, typical dosages range from about 0.01 to about 150 mg/kg/day, more typically from about 0.1 to about 15 mg/kg/day, and most typically from about 1 to about 10 mg/kg/day.

For other modes of administration, dosage amount and interval can be adjusted individually to provide plasma levels of the administered compound effective for the particular clinical indication being treated. For example, if acute inflammatory processes are the most dominant clinical manifestation, in one embodiment, a compound according to the invention can be administered in relatively high concentrations multiple times per day. Alternatively, if the patient exhibits only periodic inflammatory crises on an infrequent, periodic or irregular basis, in one embodiment, it may be more desirable to administer a compound of the invention at minimal effective concentrations and to use a less frequent administration regimen. This will provide a therapeutic regimen that is commensurate with the severity of the individual's inflammatory disease.

Utilizing the teachings provided herein, an effective prophylactic or therapeutic treatment regimen can be planned which does not cause substantial toxicity and yet is entirely effective to treat the clinical symptoms demonstrated by the particular patient. This planning should involve the careful choice of active compound by considering factors such as compound potency, relative bioavailability, patient body weight, presence and severity of adverse side effects, preferred mode of administration and the toxicity profile of the selected agent.

Compound Toxicity

The ratio between toxicity and therapeutic effect for a particular compound is its therapeutic index and can be expressed as the ratio between LD50 (the amount of compound lethal in 50% of the population) and ED50 (the amount of compound effective in 50% of the population). Compounds that exhibit high therapeutic indices are preferred. Therapeutic index data obtained from cell culture assays and/or animal studies can be used in formulating a range of dosages for use in humans. The dosage of such compounds preferably lies within a range of plasma concentrations that include the ED50 with little or no toxicity. The dosage may vary within this range depending upon the dosage form employed and the route of administration utilized. See, e.g., In The Pharmacological Basis of Therapeutics, Ch.1, p. 1, 1975. The exact formulation, route of administration and dosage can be chosen by the individual physician in view of the patient's condition and the particular method in which the compound is used.

Methods

In addition to the compounds and pharmaceutical formulations discussed in detail above, the present invention provides a number of methods in which the compounds of the invention find use. The methods range from those that might be used in a laboratory setting to probe the basic mechanisms of, for example, pharmacokinetics, drug activity, disease origin and progression and the like.

The invention is particularly useful in treating or preventing inflammatory diseases. An “inflammatory process” as used herein is a disease in which lymphoproliferation contributes to tissue or organ damage leading to disease. For instance, excessive T-cell proliferation at the site of a tissue or organ will cause damage to the tissue or organ. Inflammatory processes are well known in the art and have been described extensively in medical textbooks (See, e.g., Harrison's Principles of Experimental Medicine, 13th Edition, McGraw-Hill, Inc., N.Y.).

In an exemplary embodiment, the present invention provides a method for treating or preventing an inflammatory process, involving administering to a subject suffering from an inflammatory process a therapeutically effective amount of a compound having the structure according to Formula I.

Disease associated with abnormalities of the inflammatory process include but are not limited to proliferative glomerulonephritis; lupus erythematosus; scleroderma; temporal arteritis; thromboangiitis obliterans; mucocutaneous lymph node syndrome; asthma; host versus graft syndrome; inflammatory bowel disease; cancer; multiple sclerosis; rheumatoid arthritis; thyroiditis; Grave's disease; antigen-induced airway hyperactivity; pulmonary eosinophilia; Guillain-Barre syndrome; allergic rhinitis; myasthenia gravis; human T-lymphotrophic virus type 1-associated myelopathy; herpes simplex encephalitis; inflammatory myopathies; atherosclerosis; Goodpasture's syndrome, insulin-dependent (Type 1) diabetes mellitus, peripheral neuritis, experimental autoimmune myocarditis and pulmonary hypertension. Some examples of inflammatory processes as well as animal models for testing and developing the compounds are set forth in Table 2 below.

In addition to the particular processes enumerated above, the invention is also useful in treating or preventing dermatological diseases including keloids, hypertrophic scars, seborrheic dermatosis, papilloma virus infection (e.g., producing verruca vulgaris, verruca plantaris, verruca plan, condylomata, etc.), eczema, Karposi's sarcoma, and epithelial precancerous lesions such as actinic keratosis.

TABLE 2

| Disease | Proliferating Cells | Reference | Animal Model | Reference |
|---|---|---|---|---|
| Asthma | T-cells | Hogg 1997 | Airway | Henderson et al. 1997 |
| | | APMIS | inflammation and | J Clin Invest 100(12) |
| | | 100: 105(10) | hyper- | 3083-3092 |
| | | | responsiveness in | |
| | | | Ovalbumisensitized | |
| | | | mice or guinea | |
| | | | pigs. | |
| Glomeru- | Mesangial | Nitta et al. 1997 | NZB/NZW crossed | Clynes et al. 1998 |
| lonephritis | (glomerular) cells | Eur J. Pharmacol | mice develop | Science 279 |
| | | 344: 107-110 | glomerular disease | (5353): 1052-54. |
| | | | and lupus-like | |
| | | | syndrome. | |
| Host versus Graft | T-cells | Schorlemmer et al. | Renal allograft | Lazarivuts et al. 1996 |
| | B cells | 1997 Int J Tissue | rejection in mice. | Nature 380 (6576) |
| | | React 19: 157-61. | | 717-720. |
| | | J. Immunol | | |
| | | 160: 5320-30 | | |
| Inflammatory | Epithelial cells | Bajaj-Elliott et al. | Trinitro-benzene | Boughton-Smith et al. |
| Bowel Disease | | 1997 Am J. Pathol. | sulphonic acid | 1988 Br J Pharmacol |
| | | 151: 1469-76 | induced bowel | 94: 65-72. |
| | | | inflammation in | |
| | | | rats. | |
| Systemic Lupus | Glomerular cells | Kodera et al. 1997 | NZB/NZW crossed | Peng et al. 1996 Mol |
| Erythematosis | Lymphocytes | Am J Nephol | mice develop | Biol Rep 23(3-4): |
| | | 17: 466-70. | glomerular disease | 247-51. |
| | | Akashi et al. 1998 | and lupus-like | |
| | | Immunology | syndrome. | |
| | | 93: 238-48 | | |
| Multiple Sclerosis | T-cells | Constantinesecu et | Experimental | Drescher et al. 1998 J |
| | | al. 1998 Immunol | allergic | Clin Invest |
| | | Res 17(1-2): 217-27. | encephalmyelitis. | 101(8): 1765-74. |
| Rheumatoid | T-cells | Ceponis et al. 1998 | Rat adjuvant | Anderson et al. 1996 J |
| Arthritis | Synovial cells | Br J Rheumatol | arthritis assay | Clin Invest |
| | | 37(2): 170-8 | | 97(11): 2672-9. |
| Thyroiditis | T-cells and | Rose et al. 1997 | HLA transgenic | J Clin Investig |
| | Epithelial cells | Crit Rev Immunol | mice immunized | 101(5): 921-6. |
| | | 17: 511-7. | with thryoglobulin. | |
| | | Schumm-Draeger | | |
| | | et al. 1996 Verh | | |
| | | Dtsch Ges Pathol | | |
| | | 80: 297-301. | | |
| Grave's Disease | Thyroid cells | DiPaola et al. 1997 | Thiouracil-fed rats. | Vilietto et al. 1997 |
| | | J Clin Endocrinol | | Oncogene 15: 2687-98. |
| | | Metab 82: 670-3. | | |
| Antigen-induced | T-cells | Wolyniec et al. | | |
| airway | | 1998 Am J Respir | | |
| hyperactivity | | Cell Mol Biol | | |
| | | 18: 777-85 | | |
| Pulmonary | T-cells | Wolyniec et al. | | |
| eosinophilia | | 1998 Am J Respir | | |
| | | Cell Mol Biol | | |
| | | 18: 777-85 | | |
| Guillain-Barre | T-cells | Hartung et al. 1991 | Experimental | |
| Syndrome | | Ann Neurol. 30: | autoimmune | |
| (inflammatory | | 48-53 | neuritis | |
| demyelinating | | | (immunization with | |
| disease) | | | PNS myelin and | |
| | | | Freunds complete | |
| | | | adjuvant) | |
| GianT-cell arteritis | T-cells | Brack et al. 1997 | | |
| (a form of systemic | | Mol Med 3: 530-43 | | |
| vasculitis) | | | | |
| Inflammation of | | | | |
| large arteries. | | | | |
| Allergic Rhinitis | T-cells | Baraniuk et al. | | |
| | | 1997 J Allergy Clin | | |
| | | Immunol 99: S763-72 | | |
| Myasthenia gravis | T-cells | Hartung et al. 1991 | | |
| | | Ann Neurol 30: 48-53 | | |
| Human T- | T-cells | Nakamura et al. | | |
| lymphotropic virus | | 1996 Intem Mede | | |
| type 1 - associated | | 35: 195-99 | | |
| myelopathy | | | | |
| Herpes simplex | T cels | Hartung et al. 1991 | | |
| encephalitis | | Ann Neurol 30: 48-53 | | |
| Inflammatory | T-cells | Hartung et al. 1991 | | |
| myopathies (ie. | | Ann Neurol 30: 48-53 | | |
| Polymyositis, | | Lindberg et al. | | |
| dermatomyocitis) | | 1995 Scan J | | |
| | | Immunol 141: 421-26 | | |
| Artherosclerosis | T-cells | Rosenfeld et al. | | |
| | | 1996 Diabetes Res | | |
| | | Clin Pract 30 | | |
| | | suppl.: 1-11 | | |
| Goodpasture's | Macrophages | Lan et al. 1995 Am | | |
| syndrome | | J Pathol 147: 1214-20 | | |

Thus, in a first aspect, the invention provides a method for treating or preventing an inflammatory process, said method comprising administering to a subject suffering from said inflammatory process a therapeutically effective amount of a compound according to Formula (I) as set forth above.

Thus, in another aspect, the present invention provides for a method of treating or preventing multiple sclerosis. The method includes administering to a subject suffering from multiple sclerosis a therapeutically effective amount of a compound having a structure according to Formula (I). In another exemplary embodiment, the method involves treating multiple sclerosis by administering a compound of the invention to a mammal not otherwise in need of treatment with the compounds of the invention.

In another aspect, the present invention provides a method of treating or preventing pulmonary hypertension. The method includes administering to a subject suffering from pulmonary hypertension a therapeutically effective amount of a compound having the structure according to Formula (I). In another exemplary embodiment, the method involves treating pulmonary hypertension by administering a compound of the invention to a mammal not otherwise in need of treatment with the compounds of the invention.

There is an excellent track record of treating nervous and cardiovascular disorders with ion channel modulators—either openers or blockers. Ion channel blockers as a general class, represent the major therapeutic agents for treatment of stroke, epilepsy and arrhythmias. In still a further aspect, the invention provides a method of treating or preventing stroke. The method includes administering to a subject suffering from stroke, or at risk of having a stroke, a therapeutically effective amount of a compound having the structure according to Formula (I). In an exemplary embodiment, the method involves treating or preventing stroke by administering a compound of the invention to a mammal not otherwise in need of treatment with the compounds of the invention.

In an exemplary embodiment of the methods of the invention, the subject treated using the methods does not have sickle cell disease.

In summary, the invention provides methods for treating or preventing various disease states. Accordingly, in one aspect, the invention provides a method for treating or preventing an inflammatory process. The method includes administering to a subject suffering from the inflammatory process or at risk of suffering from an inflammatory process a therapeutically effective amount of a compound according to Formula I:

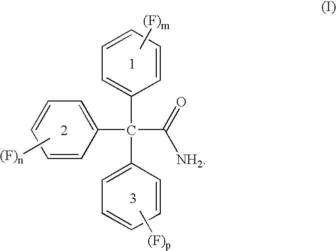

In Formula I, m, n and p are independently selected from 0 and 1 and at least one of m, n and p is 1. When m, n and p are all 1, the fluoro substituents at ring 1 and at ring 2 are located at a position independently selected from ortho to the acetamide substituent, meta to the acetamide substituent and para to the acetamide substituent, and the substituent at ring 3 is at a position selected from ortho to the acetamide substituent and para to the acetamide substituent. When p is 0, and m is 1 and n is 1, the fluoro substituent at ring 1 is para to the acetamide substituent, and the substituent at ring 2 is located at a position selected from ortho to the acetamide substituent and para to the acetamide substituent.

The invention also provides a method according to the paragraph above, wherein the disease state is selected from multiple sclerosis, insulin-dependent (type I) diabetes mellitus, rheumatoid arthritis, peripheral neuritis and pulmonary hypertension.

The present invention also provides a method for treating or preventing multiple sclerosis. The method includes administering to a subject suffering from multiple sclerosis or at risk of developing multiple sclerosis a therapeutically effective amount of a compound according to Formula I.

Also provided is a method for treating or preventing pulmonary hypertension. The method comprising administering to a subject suffering from pulmonary hypertension a therapeutically effective amount of a compound according to Formula I:

The invention further provides a method for treating or preventing a stroke. The method includes administering to a subject suffering from a stroke or at risk of having a stroke a therapeutically effective amount of a compound according to Formula I

Also provided is a method according to any of the paragraph above, wherein the compound has a structure according to Formula II:

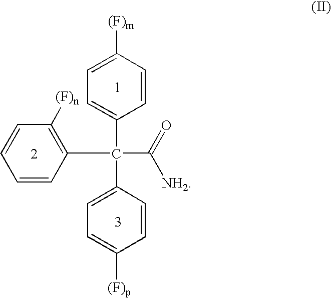

In Formula II, m, n and p are independently selected from 0 and 1, and at least one of m,nandpis 1.

Another embodiment provides a method according to any of the paragraphs above, wherein the compound has a structure according to Formula III:

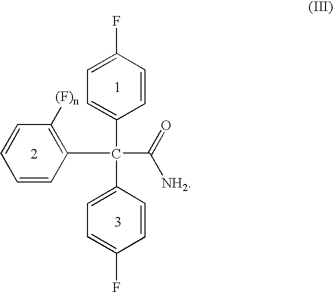

In Formula III, n is an integer selected from 0 and 1.

Also provided is a method according to any of the paragraphs above in which the compound has a structure that is selected from:

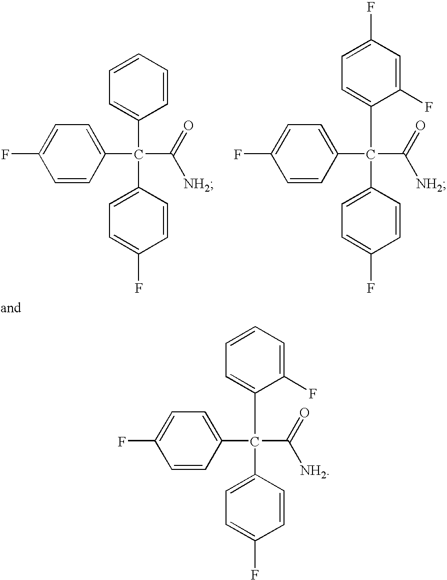

The invention also provides a method of any of the paragraphs above, wherein the disease state is mediated by a potassium channel.

In an exemplary method according to of any of the paragraphs above, the potassium channel is IK1.

In an exemplary embodiment according to any of the paragraphs above, the subject treated using the method set forth in any of the paragraphs above does not have sickle cell disease.

The compounds, compositions and methods of the present invention are further illustrated by the examples that follow. These examples are offered to illustrate, but not to limit the claimed invention.

### EXAMPLES

Example 1 illustrates methods for the synthesis and characterization of compounds of the invention. The compounds of the invention were isolated in substantially pure form and in good yields utilizing the methods detailed in this Example. Other synthetic methods are disclosed in U.S. Pat. No. 6,288,122 and U.S. Pat. No. 6,028,103.

Example 2 describes a bioassay for measuring the inhibition of potassium channel by the compounds of the invention.

### Example 1

This Example illustrates methods for the synthesis and characterization of compounds of the invention. The compounds of the invention were isolated in substantially pure form and in good yields utilizing the methods detailed below. The example provides methods of general scope that can be used to synthesize compounds of the invention other than those specifically exemplified.

1.1 Materials and Methods

Reagents were used as received unless otherwise stated. The method of Franco et al., J. Chem. Soc. Perkins Trans. II, 443 (1988), was used to prepare non-commercial fluorophenyllithium reagents and fluorobenzophenones. All moisture-sensitive reactions were performed under a nitrogen atmosphere using oven dried glassware. Reactions were monitored by TLC on silica gel 60 F₂₅₄ with detection by charring with Hancssian's stain (Khadem et al., Anal. Chem., 30: 1958 (1965)). Column chromatography was carried out using Selecto silica gel (32-63 Γ M). Melting points were determined on an Electrothermal IA9000 unit and are uncorrected. ¹H (300 MHz) and ¹⁹F (282 MHz) spectra were recorded on a Varian (Gemini 2000) NMR machine at room temperature in CDCl₃. Tetramethylsilane was used as the internal reference. Chiral separation of compound 1 was performed by Chiral Technologies using a CHLIRACELl toreq. OD-R column and acetonitrile/water as the eluant.

1.2 Preparation of Compound 1

Compound 1 was prepared in 28% yield in four steps from commercially available precursors.
1.2a Synthesis of (2-fluorophenyl)-(4-fluorophenyl)phenylmethanol

Phenylmagnesium bromide (1.83 mL, 5.5 mmol) was added dropwise to a stirring solution of 2,4′-difluorobenzophenone (1.09 g, 5.0 mmol) in t-butylmethyl ether (12 mL) at room temperature (“rt,” about 25 ° C.). After the addition was complete the reaction was heated at reflux for 3 h. The solution was cooled to rt and was poured in to ice cold 1.0 M HCl (aq) (20 mL). The organics were extracted with EtOAc (3×10mL) and dried (Na₂SO₄). Concentration under reduced pressure gave the desired product (2-fluorophenyl)-(4-fluorophenyl) phenylmethanol as a pale brown oil which was used in the next reaction without any further purification.
1.2b Synthesis of (2-fluorophenyl)-(4-fluorophenyl)phenylacetonitrile

(2-Fluorophenyl)-(4-fluorophenyl)phenylmethanol (1.47 g, 5.0 mmol) was added to a 20% solution of acetyl chloride in dichloromethane (10 mL) at rt. The resulting solution was stirred for 12 h after which the solvent was removed by evaporation. Toluene (2×20 mL) was added to the residue and evaporated to afford crude 2-fluorophenyl-(4-fluorophenyl)phenylchloromethane which was used without purification in the next step.

Copper cyanide (0.50 g, 5.5 mmol) was added to the residue and the resultant mixture was heated at 130 ° C. for 2.5 h. Once the reaction had cooled to approximately 110° C. toluene (30 mL) was added and the mixture was stirred vigorously for 10 min. The mixture was filtered and the solvent was removed under reduced pressure. Hot hexane (30 mL) was added to the crude material and the mixture was stirred vigorously for 30 min. Filtration and washing with more hexane gave the desired cyano product as a white solid, which was used without further purification.
1.2c Synthesis of (2-fluorophenyl)-(4-fluorophenyl)phenylacetamide (1)

A solution of concentrated sulfuric acid (10 mL) and glacial acetic acid (10 mL) was added to crude (2-fluorophenyl)-(4-fluorophenyl)phenylacctonitrile (1.48 g, 5.0 mmol) at rt. The resulting orange solution was stirred and heated at 130° C. for 3 h. The reaction was cooled to 0° C. and was neutralized by the dropwise addition of ammonium hydroxide. Water was added (30 mL) and the organics were extracted with chloroform (3×30 mL). The organic fractions were combined and washed sequentially with water (2×10 mL) and brine (20 mL). The organic phase was dried (Na₂SO₄) and concentrated under reduced pressure. Hexane (30 mL) was added to the resulting light brown oil to initiate precipitation. The precipitate was ground up and washed sequentially with hot hexane (30 mL). Crystallization from hexane/dichloromethane gave the desired product (2-fluorophenyl)-(4-fluorophenyl)phenylacetamide as a white crystalline solid (0.45 g, 1.4 mmol, 28%, 4 steps).

1.3 Preparation of Compound 3

Compound 3 was prepared in three steps from commercially available precursors in 58% yield.
1.3a Synthesis of bis(4-fluorophenyl)phenylmethanol

Phenylmagnesium bromide (100 mL,0.1 mol) was added dropwise to a stirring solution of 4,4′-difluorobenzophenone (20 g, 0.092 mol) in t-butylmethyl ether (150 mL) at rt. After the addition was complete the reaction was heated at reflux for 3 h. The solution was cooled to rt and was poured in to ice cold aqueous 1.0 M HCl (100 mL). The organics were extracted with EtOAc (2×50 mL) and dried (Na₂SO₄). Concentration under reduced pressure gave bis(4-fluorophenyl)phenylmethanol as a pale brown oil. After drying in vacuo for 2 h the crude material was used in the next reaction without any further purification.
1.3b Synthesis of bis(4-fluorophenyl)phenylacetonitrile

Bis(4-fluorophenyl)phenylmethanol (0.092 mol) was added to a 20% solution of acetyl chloride in dichloromethane (50 mL) at rt. The resulting purple solution was stirred for 12 h after which the solvent was removed by evaporation. Toluene (100 mL) was added to the residue and then evaporated, affording crude bis(4-fluorophenyl)phenylchloromethane which was used without purification in the following step.

Copper cyanide (8.24 g, 0.11 mol) was added to the crude residue and the mixture was heated at 140° C. for 3 h. The reaction was cooled to 100° C. and toluene (100 mL) was added. The resulting mixture was stirred vigorously for 10 min, cooled to rt, filtered through a short pad of silica and the solvent was removed under reduced pressure to afford a brown solid. Hot hexane (100 mL) was added to the powdered crude material and the mixture was stirred vigorously for 4 h. Filtration and washing with additional hexane gave the desired bis(4-fluorophenyl)phenylacetonitrile as a white solid (18.9 g, 67%).
1.3c Synthesis of bis(4-fluorophenyl)phenylacetamide (3)

A solution of concentrated sulfuric acid (50 mL) and glacial acetic acid (50 mL) was added to bis(4-fluorophenyl)phenylacetonitrile (18.9 g, 0.06 mol) at rt. The resulting orange solution was stirred and heated at 130° C. for 3 h. The reaction was cooled to 0° C., poured into ice water (150 mL) and neutralized with ammonium hydroxide. The organics were extracted with chloroform (3×100 mL), combined and washed with brine (2×50 mL). The organics were dried (Na₂SO₄) and concentrated under reduced pressure to afford a yellow-orange solid. The solid was stirred with hot hexane (100 ml) for 30 min and filtered. Crystallization from dichloromethane/hexane gave bis(4-fluorophenyl)phenylacetamide (3) as a white crystalline solid (16.9 g, 0.052 mol, 87%).

1.4 Preparation of Compound 5

Compound 5 was prepared in 66% yield in four steps from commercially available precursors.

1.4a Synthesis of bis(4-fluorophenyl)-2-fluorophenylmethanol

p-Fluorophenylmagnesium bromide (124 mL, 0.12 mol) was added dropwise to a stirring solution of 2,4′-difluorobenzophenone (24.5 g, 0.11 mol) in t-butylmethyl ether (100 mL) at rt. After the addition was complete the reaction was heated at reflux for 3 h. The solution was then cooled to rt and was poured in to ice cold 1.0 M HCl (aq) (100 mL). The organics were extracted with EtOAc (3×70 mL) and dried (Na₂SO₄). Concentration under reduced pressure gave the desired product bis(4-fluorophenyl)-2-fluorophenylmethanol as a pale yellow oil which was used in the next reaction without any further purification.
1.4b Synthesis of bis(4-fluorophenyl)-2-fluorophenylacetonitrile

A 20% solution of acetyl chloride in dichloromethane (60 mL) was added to the crude bis(4-fluorophenyl)-2-fluorophenylmethanol at rt. The resulting solution was stirred for 12 h after which the solvent was removed by evaporation. Toluene (100 mL) was added to the residue and was then evaporated to afford crude bis(4-fluorophenyl)-2-fluorophenylchloromethane which was used without purification in the next step.

Copper cyanide (12 g, 0.13 mol) was added to the crude material and the resulting mixture was heated at 160° C. for 3 h. The reaction was cooled to approximately 110° C., toluene (100 mL) was added and the mixture was stirred vigorously for 10 min. The mixture was cooled, filtered through a short silica plug and concentrated under reduced pressure. Hot hexane (100 mL) was added to the crude material and the mixture was stirred vigorously for 30 min. Filtration and washing with more hexane gave the desired bis(4-fluorophenyl)-2-fluorophenylacetonitrile as a white solid (25.3 g, 70%).
1.4c Synthesis of bis(4-fluorophenyl)-2-fluorophenylacetamide (5)

A solution of concentrated sulfuric acid (10 mL) and glacial acetic acid (10 mL) was added to bis(4-fluorophenyl)-2-fluorophenylacetonitrilc (5.0 g, 0.015 mol) at rt. The resulting orange solution was stirred and heated at 130° C. for 2 h. The reaction was cooled to 0° C. and was poured onto ice (50 g). The resulting mixture was neutralized by the dropwise addition of ammonium hydroxide. Methylene chloride (100 mL) was added and the organics were extracted with additional methylene chloride (3×30 mL). The combined organic fractions were washed sequentially with water (2×10 mL) and brine (20 mL). The organic phase was dried (Na₂SO4) and concentrated under reduced pressure to afford a yellow/orange solid. The solid was powdered and washed repeatedly with hot hexane (50 ml) until no coloration was evident in the filtrate. Crystallization from hexane/dichloromethane gave the desired product bis(4-fluorophenyl)-2-fluorophenylacetamide 5 as a white crystalline solid (4.98 g, 0.0145 mol, 94%).

1.5 Preparation of Compound 16

Compound 16 was prepared in 11% yield in four steps from commercially available precursors.
1.5a Synthesis of bis(4-fluorophenyl)-3-fluorophenylmethanol

n-Butyllithium (4 mL, 10 mmol) was added dropwise to a stirring solution of bromo-3-fluorobenzene (1.75 g, 10 mmol) in THF (25 mL) at −78° C. After 20 min 4,4′-benzophenone (1.96 g, 9 mmol) was added. The reaction was allowed to warm to 0° C. over a 30 min period. Saturated ammonium chloride (aq) (30 mL) was added and stirring was continued for 30 min. EtOAc (20 mL) was added, the organics were separated, washed with brine (20 mL), dried (Na₂SO₄) and concentrated under reduced pressure. The residue was purified by column chromatography (100% hexane to 100% methylene chloride) to afford bis(4-fluorophenyl)-3-fluorophenylmethanol (2.81 g, 92%).
1.5b Synthesis of bis(4-fluorophenyl)-3-fluorophenylacetonitrile

Bis(4-fluorophenyl)-3-fluorophenylmethanol (999 mg, 3.18 mmol) was added to a 20% solution of acetyl chloride in dichloromethane (10 mL) at rt. The resulting purple solution was stirred for 12 h after which the solvent was removed by evaporation. Toluene (20 mL) was added to the residue and then evaporated affording crude bis(4-fluorophenyl)-3-fluorophenylchloromethane which was used in the next step without purification.

Copper cyanide (344 mg, 3.82 mmol) was added to the crude material and the resulting mixture was heated at 140° C. for 3 h. The reaction was cooled to approximately 110° C., toluene (50 mL) was added and the mixture was stirred vigorously for 10 min. The mixture was cooled to rt, filtered through a short pad of silica and the solvent was removed under reduced pressure to afford a beige solid. Hot hexane (100 mL) was added to the powdered crude material and the mixture was stirred vigorously for 1 h. Filtration and washing with additional hexane gave bis(4-fluorophenyl)-3-fluorophenylacetonitrile as a white solid which was used without further purification.
1.5c Synthesis of bis(4-fluorophenyl)-3-fluorophenylacetamide (16)

A solution of concentrated sulfuric acid (10 mL) and glacial acetic acid (10 mL) was added to bis(4-fluorophenyl)-3-fluorophenylacetonitrile (3.18 mmol) at rt. The resulting orange solution was stirred and heated at 130° C. for 3 h. The reaction was cooled to 0° C., poured into ice water (50 mL) and neutralized with ammonium hydroxide. The organics were extracted with chloroform (3×50 mL). The organics fractions were combined washed with brine (2×20 mL), dried (Na₂SO₄) and concentrated under reduced pressure to afford a yellow-orange solid. The solid was stirred with hot hexane (50 ml) for 30 min and filtered. Crystallization from dichloromethane/hexane gave the desired product bis(4-fluorophenyl)-3-fluorophenylacetamide 16 as a white crystalline solid (147 mg, 0.43 mmol, 1 1%, 4 steps).

1.6 Compound Characterization by ¹H and ¹⁹F NMR Spectroscopy and Melting Point

The compounds of the invention were characterized by a combination of ¹H and ¹⁹F NMR spectroscopy and the compound melting points were determined.

1: ¹H NMR Γ (CHCl₃): 7.39-7.26 (8H, m), 7.15-6.90 (5H, m), 5.83 (1H, brs), 5.72 (1H, brs); ¹⁹F NMR Γ (CHCl₃): −103.4 (1F, s), −115.8 (1F, s); m.p 180-181° C.

2: ¹H NMR Γ (CHCl₃): 7.37-7.28 (6H, m), 7.15-7.05 (2H, m), 6.93 (1H, dt, J=8 and 2 Hz), 5.90 (1H, brs), 5.68 (1H, brs); ¹⁹F NMR Γ (CHCl₃): −103.4 (1F, m); m.p 210° C.

3: ¹H NMR Γ (CHCl₃): 7.37-7.20 (9H, m), 7.04-6.91 (4H, m), 5.81 (1H, brs), 5.71 (1H, brs); ¹⁹F NMR Γ (CHCl₃): −115.7 (2F, s); m.p 180-181° C.

4: ¹H NMR Γ (CHCl₃): 7.37-7.24 (12H, m), 6.97 (2H, t, J=8.5 Hz), 5.83 (1H, brs), 5.75 (1H, brs); ¹⁹F NMR Γ (CHCl₃): −116.2 (1F, s); m.p 193-194° C.

5: ¹H NMR Γ (CHCl₃): 7.41-7.34 (1H, m), 7.29-7.23 (4H, m), 7.16 (1H, ddd, J=18.1, 8.1 and 1.2 Hz), 7.15 (1H, d, J=7.7 Hz), 7.05-6.97 (4H,m), 6.93-6.87 (1H, dt, J=8.0 and 1.4 Hz), 5.90 (1H, brs), 5.74 (1H, brs); ¹⁹F NMR Γ (CHCl₃): −103.3 (1F, s), −115.5 (2F, s); m.p 168-169° C.

6: ¹H NMR Γ (CHCl₃): 7.64-7.54 (4H, m), 7.40-7.34 (6H, m), 5.70 (2H, brs); ¹⁹F NMR Γ (CHCl₃): 137.3 (2F, d, J=19.2 Hz), −155.8 (1F, t, J=21.4 Hz), −161.9 (2F, dd, J=21.4 and 17.1 Hz).

7: ¹H NMR Γ (CHCl₃): 7.37-7.31 (6H, m), 7.28-7.20 (5H, m), 7.12-7.04 (2H, m), 5.90 ((1H, brs), 5.74 (1H, brs); . ¹⁹F NMR Γ (CHCl₃): −137.9 (1F, m), −140.3 to −140.4 (1F, m); m.p 174-175° C.

8: ¹H NMR Γ (CHCl₃): 7.37-7.28 (10H, m), 6.95-6.83 (2H, m), 6.81-6.75 (1H, m), 5.92 (1H, brs), 5.80 (H, brs); ¹⁹F NMR Γ (CHCl₃): −99.1 (1F, dd, J=19.2 and 8.5 Hz), −111.6 (1F, m); m.p 187-188° C.

9: ¹H NMR Γ (CHCl₃): 7.38-7.22 (7H, m), 7.09-6.96 (6H, m), 5.83 (1H, brs), 5.77 (1H, brs);. ¹⁹F NMR Γ (CHCl₃): −112.6 (2F, dd, J=17.1 and 6.4 Hz); m.p 195-196° C.

13: ¹H NMR Γ (CHCl₃): 7.26-7.19 (6H, dd, J=9.0 and 5.4 Hz), 7.20-7.01 (6H, t, J=8.7 Hz), 5.83 (1H, brs), 5.69 (1H, brs);. ¹⁹F NMR Γ (CHCl₃): -115.3 (3F, s); m.p 180-181° C.

14: ¹H NMR Γ (CHCl₃): 7.39-7.27 (9H, m), 7.17-7.03 (4H, m), 5.90 (1H, brs), 5.85 (1H, brs);. ¹⁹F NMR Γ (CHCl₃): −102.9 (2F, s); m.p 166-167° C.

15: ¹H NMR Γ (CHCl₃): 7.41-7.34 (2H, m), 7.29-7.23 (4H, m), 7.17-7.05 (4H, m), 6.99 (2H, t, J=8.7 Hz), 5.78(2H, brs); ¹⁹F NMR Γ (CHCl₃): −103.0 (2F, s), −115.9 (1F, m); m.p 187-188° C.

16: ¹H NMR Γ (CHCl₃): 7.34-7.20 (6H, m), 7.06-6.97 (6H, m), 5.90 (1H, brs), 5.71 (1H, brs); ¹⁹F NMR Γ (CHCl₃): −112.2 (1F, dd, J=17.1 and 7.4 Hz), =115.1 to -115.2 (2F, m); m.p 165-166° C.

17: ¹H NMR Γ (CHCl₃): 7.35 -7.21 (3H, m), 7.06-6.97 (9H, m), 7.17-7.05 (4H, m), 5.96 (1H, brs), 5.76 (1H, brs); ¹⁹F NMR Γ (CHCl₃): −112.2 (3F, dd, J=17.1 and 8.5 Hz); m.p 186-188° C.

### Example 2

Studies for the Treatment of MS

The effect of IK1 blockers on multiple sclerosis can be examined in mice. Exemplary mice of use are female C57BL/6 mice. EAE is first induced in the mice and then treated with the IK1 blocker. For EAE induction, 150 μg of MOG₃₅₋₅₅ peptide and 300 μg of killed Mycobacterium tuberculosis can be mixed in CFA and injected s.c. in two 50 μl injections over the flanks of the mice on day 1. Also, 200 ng of pertussis toxin can be injected i.v. on days 0 and 2. The animals are anesthetized by isoflurane inhalation.

The IK1 blockers of the invention can be introduced in a formulation and administered twice daily in a 100 μl volume by i.p. injection into the mice. An example of a formulation includes the IK1 blocker in saline and 0.4% methylcellulose. Dosing with an IK1 blocker starts at day 0, 24 h prior to MOG₃₅₋₅₅ immunization (day 1). Mice are then monitored daily and assessed for clinical signs of disease in a blinded fashion. The following criteria can be used to determine the symptoms of multiple sclerosis: 0, no signs of disease; 1, tail paralysis; 2, limp tail and hind limb weakness; 3, hind limb paralysis; 4, hind limb plus forelimb paralysis; and 5, moribund or dead. Cumulative clinical scores can be calculated by adding daily scores from the day of immunization until the end of the experiment. Mean clinical scores at separate days and mean maximal scores can be calculated by adding the scores of individual mice and dividing with the number of mice in each group, including mice not developing signs of EAE.

For immunohistochemical analysis of the mice, the following antibodies can be used: anti-CD4, anti-CD152, anti-ICOS, anti-mouse-IFN-γ, and anti-mouse-TNF-α. Biotinylated rabbit anti-rat-IgG (H+L), and biotinylated antihamster-IgG (H+L) can also be used.

At the termination of experiment, the mice are perfused with saline through the left ventricle of the heart. Brains and spinal cords can then be dissected and the spinal cord segments can be embedded in OCT medium and frozen. H&E staining can then be performed to examine the cell infiltration of spinal cords. Peroxidase-based immunohistochemical staining can also be performed to determine various cell types in the lesions of the spinal cord. To accomplish this, spinal cord sections can be incubated with one of the primary antibodies to mouse CD4 and ICOS, or isotype-control mAb, followed by biotin-conjugated second antibodies and streptavidin-HRP. For cytokine detection, the specimens can be stained with primary antibodies specific for mouse IFN-γ or mouse TNF-α. Finally, DAB can be used to develop a brown color in positively stained cells, and the tissues can be counterstained with hematoxylin.

Antigen-specific T-cell proliferation assays can then be performed by the following process. Splenocytes isolated from mice at termination can be washed with saline followed by culturing with a material supplemented with the MOG peptide. For a nonspecific-stimulation control, the cells can be incubated with Con A. The cells are cultured in 96-well microtiter plates at a density of 1×106 cells per ml. After incubation, the cells can be pulsed with ³[H] thymidine at 1 μCi per well for 24 h, then harvested and counted.

RNA can be isolated from the spinal cord tissue of individual mice using TRI-reagent. RNA integrity and concentration can be determined with an RNA 6000 Nano LapChip kit. Reverse transcription can then be carried out using a RT-PCR kit to produce cDNA copies of the RNA. Reverse transcription can be carried out as follows using the SuperScript™ first-string synthesis System for RT-PCR kit. Total RNA can be annealed with 0.5 μg of Oligo(dT) and 50 ng of random hexamers in a total volume of 12 μl at 70° C. for 10 min and chilled on ice. Then 8 μl of a cocktail containing 2.5×RT buffer, 6.25 mM MgCl₂, 1.25 mM dNTP, 25 mM DTT and 200 U SuperScript II reverse transcriptase can be added. The mixture can be incubated at 25° C. for 10 min, 42° C. for 50 min, and 70° C. for 15 min, and chilled on ice. The sample can be treated with 2 U of RNase H at 37° C for 20 mm.

Real-time PCR can then be performed on the cDNA in order to have cDNA in amounts necessary to calculate the mRNA levels in the mice. Real-time PCR can be carried out on the GeneAmp, 5700 Sequence Detection System utilizing SYBR® Green PCR Master Mix as described below. Oligonucleotides can be purchased from Invitrogen. The PCR reaction consists of 25 ng cDNA, 400 nM each target primer, and 1×final concentration of SYBR Green PCR Master Mix in a total volume of 30 μl. The following amplification parameters can be used: 50° C. for 2 min, followed by 95° C. for 10 min, and 40 cycles of 95° C. for 15 sec and 60° C. for 1 min. The reaction proceeds for another 20 min at 60° C. to determine the specificity of the primers and potential primer dimers. Samples can be run in duplicate. The mRNA levels are then calculated by using a modification of the comparative cycle threshold (C_{T}) method (Applied Biosystems' User Bulletin No. 2), with a formula of 2(^{-Δ° C.t)}×10000. The values are then normalized to the level of the corresponding ubiquitin housekeeping gene. The results are then presented for the mice with EAE who were, and were not, treated with an IK1 blocker.

A second group of treated animals can also be used for blood collection to determine the concentration of IK1 blocker in the plasma. Blood was collected by retro-orbital sinus puncture following anesthesia at 1 h, 2 h, 4 h, 6 h, 8 h, and 24 h after the last treatment. Blood is then collected into heparinized tubes and kept on ice. Plasma can be obtained by centrifugation and stored at −80° C. pending analysis. IK1 blocker concentration in plasma is determined using LC-MS/MS. An example of a mass spectrometer of use in the experiment is a Waters/Micromass Micro triple quadrupole. Sample introduction into the LC-MS/MS can be carried out using a CTC HTS-PAL autosampler with a four-way Harney valve and random sampling capabilities. The HPLC system can also include two Shimadzu LC-10ADvp pumps and a Luna C18(2) 2.0×50.0 mm, 5 μM column.

A gradient elution using solvent mixtures A (SMA) and B (SMB) at a flow rate of 0.25 ml/min can be employed. SMA is 0.1% formic acid in 95% aqueous methanol and SMB is 0.1% formic acid in 5% aqueous methanol. The gradient conditions are: for the first 1.5 min after injection, 100% A; from 1.5-2.5 min, 70% A; from 2.5-3.5 min, 50% A; from 3.5-4.5 min, 0% A; and at 4.5 min switched back to initial conditions (100% SMA). Plasma samples are treated with two volumes of acetonitrile, vortexed and centrifuged to precipitate the protein. The supernatant can be injected into the LC-MS/MS system. Analysis can be carried out using multiple reaction monitoring (MRM) in the positive ion mode. The transition to be monitored is m/z 345 to 277. The plasma concentrations of IK1 blockers are calculated with a five- point calibration curve prepared in plasma of undosed mice.

In vivo effects of these IK1 blockers on the progression of multiple sclerosis in mice can also be tested by using the model described in example 1 of Elloso et al., U.S. Pat. Pub. No. 2004/0167112 (Pub. Date: Aug. 26, 2004) (“Elloso”). The in vitro effect of these IK1 blockers on the inhibition of cytokine production can be tested by using the model described in example 2 of Elloso.

It is understood that the examples and embodiments described herein are for illustrative purposes only and that various modifications or changes in light thereof will be suggested to persons skilled in the art and are to be considered included within the spirit and purview of this application and are considered within the scope of the appended claims. All publications, patents, and patent applications cited herein are hereby incorporated by reference in their entirety for all purposes.

What is claimed is:

1. A method for treating or preventing an inflammatory process, said method comprising administering to a subject suffering from said inflammatory process a therapeutically effective amount of a compound according to Formula I: 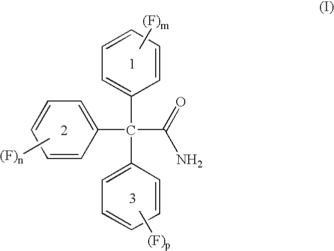
wherein,
m, n and p are independently selected from 0 and 1 and at least one of m, n and p is 1;
when m, n and p are all 1, the fluoro substituents at ring 1 and at ring 2 are located at a position independently selected from ortho to the acetamide substituent, meta to the acetamide substituent and para to the acetamide substituent, and the substituent at ring 3 is at a position selected from ortho to the acetamide substituent and para to the acetamide substituent; and
when p is 0, and m is 1 and n is 1, the fluoro substituent at ring 1 is para to the acetamide substituent, and the substituent at ring 2 is located at a position selected from ortho to the acetamide substituent and para to the acetamide substituent.

2. The method according to claim 1, wherein said compound has a structure according to Formula II: 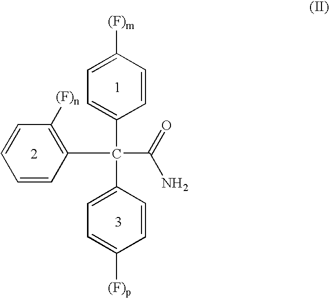
wherein
m, n and p are independently selected from 0 and 1, and at least one of m, n and p is 1.

3. The method according to claim 2, wherein said compound has a structure according to Formula III: 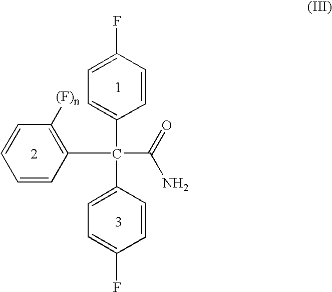
wherein
n is an integer selected from 0 and 1.

4. The method according to claim 1, said compound having a structure that is selected from:

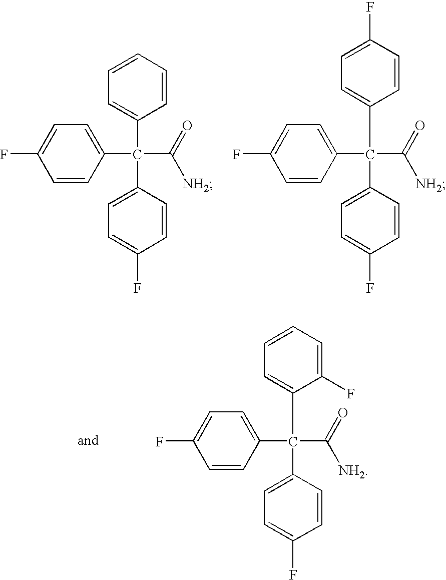

5. The method of claim 1, wherein said inflammatory process is a member selected from multiple sclerosis, insulin-dependent (type I) diabetes mellitus, rheumatoid arthritis, peripheral neuritis and pulmonary hypertension.

6. The method of claim 5, wherein said inflammatory process is multiple sclerosis.

7. The method of claim 5, wherein said inflammatory process is pulmonary hypertension.

8. The method of claim 1, wherein said inflammatory process is mediated by a potassium channel.

9. The method of claim 8, wherein said potassium channel is IK1.

10. A method for treating or preventing multiple sclerosis, said method comprising administering to a subject suffering from multiple sclerosis a therapeutically effective amount of a compound according to Formula I: 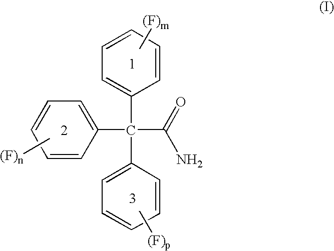
wherein,
m, n and p are independently selected from 0 and 1 and at least one of m, n and p is 1;
when m, n and p are all 1, the fluoro substituents at ring 1 and at ring 2 are located at a position independently selected from ortho to the acetamide substituent, meta to the acetamide substituent and para to the acetamide substituent, and the substituent at ring 3 is at a position selected from ortho to the acetamide substituent and para to the acetamide substituent; and
when p is 0, and m is 1 and n is 1, the fluoro substituent at ring 1 is para to the acetamide substituent, and the substituent at ring 2 is located at a position selected from ortho to the acetamide substituent and para to the acetamide substituent.

11. A method for treating or preventing pulmonary hypertension, said method comprising administering to a subject suffering from pulmonary hypertension a therapeutically effective amount of a compound according to Formula I: 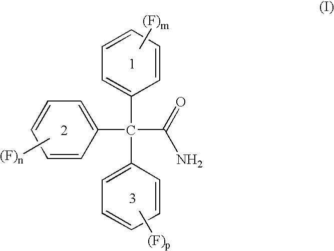
wherein,
m, n and p are independently selected from 0 and 1 and at least one of m, n and p is 1;
when m, n and p are all 1, the fluoro substituents at ring 1 and at ring 2 are located at a position independently selected from ortho to the acetamide substituent, meta to the acetamide substituent and para to the acetamide substituent, and the substituent at ring 3 is at a position selected from ortho to the acetamide substituent and para to the acetamide substituent; and
when p is 0, and m is 1 and n is 1, the fluoro substituent at ring 1 is para to the acetamide substituent, and the substituent at ring 2 is located at a position selected from ortho to the acetamide substituent and para to the acetamide substituent.

12. A method for treating or preventing a stroke, said method comprising administering to a subject suffering from a stroke or at risk of having a stroke a therapeutically effective amount of a compound according to Formula I: 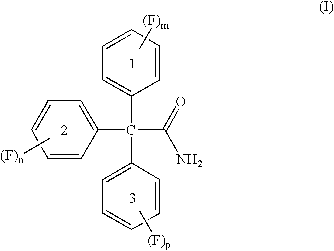
wherein,
m, n and p are independently selected from 0 and 1 and at least one of m, n and p is 1;
when m, n and p are all 1, the fluoro substituents at ring 1 and at ring 2 are located at a position independently selected from ortho to the acetamide substituent, meta to the acetamide substituent and para to the acetamide substituent, and the substituent at ring 3 is at a position selected from ortho to the acetamide substituent and para to the acetamide substituent; and
when p is 0, and m is 1 and n is 1, the fluoro substituent at ring 1 is para to the acetamide substituent, and the substituent at ring 2 is located at a position selected from ortho to the acetamide substituent and para to the acetamide substituent.

13. The method according to claim 12, wherein said compound has a structure according to Formula II: 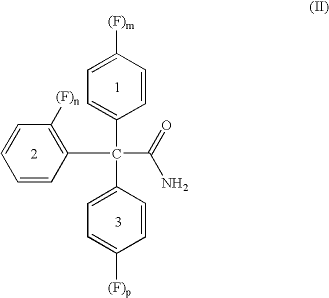
wherein
m, n and p are independently selected from 0 and 1, and at least one of m, n and p is 6 1.

14. The method according to claim 13, wherein said compound has a structure according to Formula III: 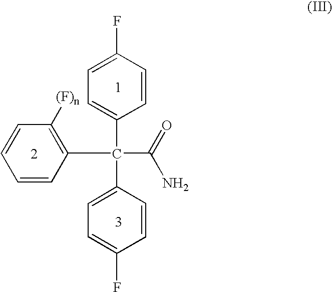
wherein
n is an integer selected from 0 and 1.

15. The method according to claim 12, said compound having a structure that is selected from: [chemical structure]
